(19) United States
(12) Patent Application Publication
(10) Pub. No.: US 2006/0004297 A1
(43) Pub. Date: Jan. 5, 2006

(54) Lung model-based cardiopulmonary performance determination

(76) Inventors: Joseph A. Orr, Park City, UT (US); Kai Kuck, Hamburg (DE); Lara Brewer, Bountiful, UT (US)

Correspondence Address: MICHAEL W. HAAS, INTELLECTUAL PROPERTY COUNSEL;RESPIRONICS, INC., 1010 MURRY RIDGE LANE, MURRYSVILLE, PA, 15668, US

(21) Appl. No.: 11/150,378

(22) Filed: Jun. 10, 2005

Related U.S. Application Data
(60) Provisional application No. 60/585,405, filed on Jul. 2, 2004.

(51) Int. Cl. A61B 5/08

(52) U.S. Cl. 600/538

(57) ABSTRACT

Methods for noninvasively evaluating indicators of cardio-pulmonary performance of a subject, such as cardiac output, pulmonary capillary blood flow, and blood carbon dioxide content, include obtaining data of an expiratory carbon dioxide signal and comparing data generated by an algorithmic lung model to the data of the expiratory carbon dioxide signal of a subject. The variables that are input into the algorithmic lung model are adjusted until the data generated thereby reflects that of the measured expiratory carbon dioxide signal with a desired degree of accuracy. Once the algorithmic lung model replicates the data of the measured expiratory carbon dioxide signal with the desired degree of accuracy, one or more of the input values may be used to determine one or more of the cardiac output, pulmonary capillary blood flow, or a blood gas content of the subject from which the expiratory carbon dioxide signal was obtained.

### CROSS-REFERENCE TO RELATED APPLICATIONS

This application claims priority under 35 U.S.C. § 119(e) from provisional U.S. patent application Ser. No. 60/585,405, filed Jul. 2, 2004 the contents of which are incorporated herein by reference.

### BACKGROUND OF THE INVENTION

1. Field of the Invention

The present invention relates to methods for noninvasively measuring cardiac output, pulmonary capillary blood flow (PCBF), and carbon dioxide levels in blood. More specifically, the present invention relates to methods that include comparing a multi-component mathematical, or algorithmic, lung model to data comprising a respiratory signal, e.g., a carbon dioxide signal, of a subject, adjusting the values that are input into the algorithmic lung model until it accurately recreates the data of the measured carbon dioxide signal, and identifying at least one of the values that were input into the algorithmic lung model to provide an estimate of cardiac output, pulmonary capillary blood flow, or blood carbon dioxide content.

2. Background of Related Art

Conventionally, indicators of the cardiopulmonary performance of a subject have been measured by invasive procedures, in which direct blood gas measurements are obtained.

Indicator dilution, an exemplary invasive, typically intermittent technique for measuring cardiac output, includes introducing a predetermined amount of an indicator into the bloodstream and through the heart of a subject. Blood downstream from the point of introduction is analyzed to determine how long it takes for the indicator to be diluted in the blood to a certain degree. From this data, a time vs. dilution curve can be obtained.

Thermodilution, in which room temperature or colder saline solution, which may be referred to as “cold” saline, is employed as the indicator, is a widely employed type of indicator dilution. The cold saline is typically introduced into the right heart bloodstream of a subject through a thermodilution catheter, which includes a thermistor at an end thereof. The thermistor is employed to measure the temperature of the blood after it has passed through the right heart, or downstream from the point at which the cold saline is introduced. A thermodilution curve, a type of time vs. dilution curve, is then generated from the data. The cardiac output of the subject may be derived from the thermodilution curve. Thermodilution and other indicator dilution techniques are, however, somewhat undesirable due to the potential for harm to the subject that is associated with inserting and maintaining such catheters in place.

To avoid the invasiveness and injury potential associated with indicator dilution procedures, less invasive techniques which rely upon parameters obtained as a subject breathes have been employed.

One of the less invasive conventional techniques for measuring the cardiac output of a subject employs the Fick principle: the rate of uptake of a substance by or release of a substance from blood at the lung is equal to the blood flow past the lung and the content difference of the substance at each side of the lung.

The Fick principle may be represented in terms of oxygen (O₂) by the following formula:
Qₜ=VO₂/(CaO₂−CvO₂), (1)
where Qₜ is the cardiac output, or blood flow, of the subject, VO₂ is the net volume of oxygen consumed by the subject per unit of time, CaO₂ is the content of O₂ in the arterial, or oxygenated, blood of the subject, and CvO₂ is the content of O₂ in the venous, or de-oxygenated, blood of the subject. The oxygen Fick principle may be employed in calculating the cardiac output of a subject either intermittently or continuously.

An exemplary, so-called “non-invasive” method of determining the cardiac output of a subject by monitoring VO₂ is disclosed in Davies et al., Continuous Fick cardiac output compared to thermodilution cardiac output, Crit. Care Med., 1986; Vol. 14, pages 881-885 (“Davies”). The method of Davies includes continually measuring the O₂ fraction of samples of gas inspired and expired by a subject, the oxygen saturation (SvO₂) of the subject's venous blood, and oxygen saturation (SaO₂) of the subject's arterial blood. The O₂ measurements are made by a metabolic gas monitor, and VO₂ calculated from these measurements. SaO₂ is measured by pulse oximetry. SvO₂ may be directly measured by a pulmonary artery (“PA”) catheter equipped to measure oxygen saturation. Each of these values is then incorporated into equation (1), the so-called “oxygen Fick equation,” to determine the cardiac output of the subject.

Although the method of Davies may be employed to intermittently or continuously determine the cardiac output of a subject, it is somewhat undesirable from the standpoint that accurate VO₂ measurements are typically difficult to obtain, especially when the subject requires an elevated fraction of inspired oxygen (FiO₂). Moreover, because the method disclosed in Davies requires continual measurement of SvO₂ with a pulmonary artery catheter, it is, in actuality, a somewhat invasive technique.

Carbon dioxide elimination (VCO₂) is the volume of carbon dioxide (CO₂) excreted from the body of a subject during respiration. Conventionally, carbon dioxide elimination has been employed as an indicator of metabolic activity. Due, in part, to the ease with which the carbon dioxide elimination (VCO₂) of a subject may be accurately measured, VCO₂ measurements are widely employed in methods of non-invasively determining the cardiac output of a subject. Because the respiratory quotient (RQ) is the ratio of carbon dioxide elimination to the amount of oxygen inhaled, VCO₂ may be substituted for VO₂ according to the following exemplary equation:
VO₂=VCO₂/RQ. (2)

An exemplary method of continuously measuring the cardiac output of a subject in terms of CO₂ is disclosed in U.S. Pat. No. 4,949,724 to Mahutte et al. (“the '724 patent”). The method of the '724 patent employs the following form of the Fick equation to determine the cardiac output of a subject:
Qₜ=VCO₂/(Hgb×RQ×(SaO₂−SvO₂)), (3)
where VCO₂/(Hgb×RQ×(SaO₂−SvO₂)) has been substituted for the VO₂/(CaO₂−CvO₂) of the oxygen Fick equation (equation (1)), and Hgb is the concentration of hemoglobin in the blood, which is typically about 13.4 g/dl. A constant, k, may be employed to replace either Hgb or Hgb×RQ.

According to the method of the '724 patent, an initial cardiac output measurement is made by thermodilution techniques. Thereafter, k is calculated. Subsequently, a CO₂ flowmeter and monitor are employed to measure VCO₂. SvO₂ is measured with a catheter and oximetry processor, and SaO₂ is measured by a pulse oximeter. The cardiac output of the subject may be continuously calculated as described above. The method of continuously measuring cardiac output of the '724 patent is, however, somewhat undesirable due to the invasiveness of using a catheter to initially determine cardiac output and to measure SvO₂ continuously, which may create additional health risks for the subject.

In order to reduce the invasiveness of techniques for determining cardiac output or pulmonary capillary blood flow and to reduce the associated potential for causing harm to a subject, carbon dioxide elimination has been used in noninvasive, so-called “rebreathing” processes, by which pulmonary capillary blood flow and cardiac output may be determined.

Rebreathing processes typically include the inhalation of a gas mixture that includes carbon dioxide. During rebreathing, the carbon dioxide elimination of the subject decreases to a level less than during normal breathing. Rebreathing during which the carbon dioxide elimination decreases to near zero is typically referred to as total rebreathing. Rebreathing that causes some decrease, but not a total cessation of carbon dioxide elimination, is typically referred to as partial rebreathing.

The carbon dioxide form of the Fick equation, which is useful with such rebreathing processes, is:
Q=VCO₂/(CvCO₂−CaCO₂), (4)
where Q is cardiac output, CvCO₂ is carbon dioxide content of the venous blood of the subject, and CaCO₂ is the carbon dioxide content of the arterial blood of the subject, has been employed to noninvasively determine the pulmonary capillary blood flow or cardiac output of a subject. The carbon dioxide elimination of the subject may be noninvasively measured as the difference per breath between the volume of carbon dioxide inhaled during inspiration and the volume of carbon dioxide exhaled during expiration, and is typically calculated as the integral of the carbon dioxide signal, or the fraction of respiratory gases that comprises carbon dioxide, or “carbon dioxide fraction,” times the rate of flow over an entire breath.

Rebreathing is useful for noninvasively estimating the carbon dioxide content of mixed venous blood (in total rebreathing) or for obviating the need to know the carbon dioxide content of the mixed venous blood (in partial rebreathing).

The partial pressure of end tidal carbon dioxide (PetCO₂ or etCO₂) is also measured in rebreathing processes. The partial pressure of end-tidal carbon dioxide, after correcting for any deadspace, is typically assumed to be approximately equal to the partial pressure of carbon dioxide in the alveoli (PACO₂) of the subject or, if there is no intrapulmonary shunt, the partial pressure of carbon dioxide in the arterial blood of the subject (PaCO₂).

Rebreathing is typically conducted with a rebreathing circuit, which causes a subject S to inhale a gas mixture that includes carbon dioxide. FIG. 1 schematically illustrates an exemplary rebreathing circuit 50 that includes a tubular airway 52 that communicates air flow to and from the lungs of a subject. Tubular airway 52 may be placed in communication with the trachea of subject S by known intubation processes, or by connection to a breathing mask positioned over the nose and/or mouth of the subject. A flow meter 72, which is typically referred to as a pneumotachometer, and a carbon dioxide sensor 74, which is typically referred to as a capnometer, are disposed between tubular airway 52 and a Y-connector 58, which may be used to connect rebreathing circuit 50 to a ventilator 55, or an end of a breathing tube that communicates with the external environment. Thus, flow meter 72 and carbon dioxide sensor 74 are exposed to any gas that flows through rebreathing circuit 50. Flow meter 72 and carbon dioxide sensor 74 communicate with at least one processing element 76, which is programmed to process signals from flow meter 72 and carbon dioxide sensor 74.

Deadspace 70 may be formed or provided along at least a portion of rebreathing circuit 50 by all or part of one or more of the elements of rebreathing circuit 50 or another breathing circuit with which rebreathing circuit 50 communicates. Although deadspace 70 is shown at a particular location on rebreathing circuit 50, it may be located elsewhere or comprise a greater portion of rebreathing circuit 50.

Alternatively, as shown in FIG. 2, an additional length of hose that communicates with hose 60 may be provided to form deadspace 70. The two ends of an additional length of hose that forms deadspace 70 are separated from one another by a two-way valve 68, which may be positioned to direct the flow of air through deadspace 70. Deadspace 70 may also include an expandable section 62. A Y-piece 58, disposed on hose 60 opposite flow meter 72 and carbon dioxide sensor 74, facilitates the connection of an inspiratory hose 54 and an expiratory hose 56 to rebreathing circuit 50 and the flow communication of the inspiratory hose 54 and expiratory hose 56 with hose 60. During inhalation, gas flows into inspiratory hose 54 from the atmosphere or a ventilator (not shown). During normal breathing, valve 68 is positioned to prevent inhaled and exhaled air from flowing through deadspace 70. During rebreathing, valve 68 is positioned to direct the flow of exhaled and inhaled gases through deadspace 70. The re-breathed air, which is inhaled from deadspace 70 during rebreathing, includes air that has been exhaled by the subject, e.g., carbon dioxide-rich air.

During total rebreathing, substantially all of the gas inhaled by the subject was expired during the previous breath. Thus, during total rebreathing, the partial pressure of end-tidal carbon dioxide (PetCO₂ or etCO₂) is typically assumed to be equal to or closely related to the partial pressure of carbon dioxide in the arterial (PaCO₂), venous (PvCO₂), or alveolar (PACO₂) blood of the subject. Total rebreathing processes are based on the assumption that neither pulmonary capillary blood flow nor the content of carbon dioxide in the venous blood of the subject (CvCO₂), changes substantially during the rebreathing process. The partial pressure of carbon dioxide in blood may be converted to the content of carbon dioxide in blood by means of a carbon dioxide dissociation curve, where the change in the carbon dioxide content of the blood (CvCO₂-CaCO₂) is equal to the slope (s) of the carbon dioxide dissociation curve multiplied by the measured change in end tidal carbon dioxide (PetCO₂), as effected by a change in effective ventilation, such as rebreathing.

In partial rebreathing, the subject inhales a mixture of “fresh” gases and gases exhaled during the previous breath. Thus, the subject does not inhale a volume of carbon dioxide as large as the volume of carbon dioxide that would be inhaled during a total rebreathing process. Conventional partial rebreathing processes typically employ a differential form of the carbon dioxide Fick equation to determine the pulmonary capillary blood flow or cardiac output of the subject, which do not require knowledge of the carbon dioxide content of the mixed venous blood. This differential form of the carbon dioxide Fick equation considers measurements of carbon dioxide elimination, CvCO₂, and the content of carbon dioxide in the alveolar blood of the subject (CACO₂) during both normal breathing and the rebreathing process as follows:

$${Q}_{BD} = \frac{{V}_{{CO}_{2 B}} - {V}_{{CO}_{2 D}}}{( {Cv CO}_{2 B} - {Cv CO}_{2 D} ) - ( {Ca CO}_{2 B} - {Ca CO}_{2 D} )} , \qquad ( 5 )$$

where Q_{BD} is cardiac output, VCO_{2B} and VCO_{2D} are the carbon dioxide production of the subject before rebreathing and during the rebreathing process, respectively, CVCO_{2B} and CVCO_{2D} are the content of CO₂ of the venous blood of the subject before rebreathing and during the rebreathing process, respectively, and CaCO_{2B} and CaCO_{2D} are the content of CO₂ in the arterial blood of the subject before rebreathing and during rebreathing, respectively. If carbon dioxide content of the alveolar blood is used in place of CaCO₂, Q_{BD} is pulmonary capillary blood flow.

Again, with a carbon dioxide dissociation curve, the measured PetCO₂ can be used to determine the change in content of carbon dioxide in the blood before and during the rebreathing process. Accordingly, the following equation can be used to determine pulmonary capillary blood flow or cardiac output when partial rebreathing is conducted:

$$Q = \frac{Δ {Vco}_{2}}{s Δ {Pet CO}_{2}} . \qquad ( 6 )$$

The term “s” in equation (6) refers to the slope of the CO₂ dissociation curve, which relates the partial pressure of CO₂ (PCO₂, typically expressed in mmHg) to the CO₂ content in blood (CxCO₂, typically expressed in ml of CO₂ per ml of blood).

Alternative differential Fick methods of measuring pulmonary capillary blood flow or cardiac output have also been employed. Such differential Fick methods typically include a brief change of PetCO₂ and VCO₂ in response to a change in effective ventilation. This brief change can be accomplished by adjusting the respiratory rate, inspiratory and/or expiratory times, or tidal volume. A brief change in effective ventilation may also be effected by adding CO₂, either directly or by rebreathing. An exemplary differential Fick method that has been employed, which is disclosed in Gedeon, A. et al. in 18 Med. & Biol. Eng. & Comput., 411-418 (1980), employs a period of increased ventilation followed immediately by a period of decreased ventilation.

The carbon dioxide elimination of a subject is typically measured over the course of a breath by the following, or an equivalent, equation:

$${VCO}_{2} = {\int }_{breath} ( \dot{V} \times {f}_{CO2} ) ⅆ t , \qquad ( 7 )$$

where V is the measured respiratory flow and f_{CO2} is the substantially simultaneously detected carbon dioxide signal, or fraction of the respiratory gases that comprises carbon dioxide or “carbon dioxide fraction.”

While state-of-the-art rebreathing processes are sometimes useful for accurately calculating indicators of a subject's cardiopulmonary performance, the calculations are based upon a number of assumptions that may be untrue. In particular, the Fick equation is based on a very simple model of the lung in which all blood and respired gases come to complete equilibrium and in which gas mixing is always ideal.

Unfortunately, gas transport in the lung is often not ideal and can only be explained by accounting for both mass-flow and diffusion within the alveoli and bronchial tree. Additionally, due to the significant volume of the lung, gases may be stored and mixed over multiple breaths.

U.S. Pat. No. 5,971,934 to Scherer et al. (the '934 patent”) describes a process that is purportedly useful for noninvasively evaluating the cardiopulmonary health of an individual and which is based upon a more complex mathematical model of the human lung—the so-called “Weibel model” of the human lung. In that process, a single expired breath, which is obtained during steady state breathing, i.e., breathing without any perturbations, is evaluated. More specifically, the slope of the very end portion of that expired breath, which is referred to in the art as the “Phase III” portion of an expirogram, is evaluated and compared with the slope of data generated by the mathematical model of the lung. If the slopes of the data do not match with the desired degree of accuracy, the cardiac output value which is put into the mathematical model of the lung is adjusted to provide the desired degree of accuracy.

The process taught by the '934 patent is somewhat undesirable from a few different perspectives. For example, cardiac output is only one of a variety of factors that determine the slope of the Phase III portion of an expirogram. As another example, when only a single breath without perturbations is evaluated, other factors that may influence the slope of the Phase III portion of an expirogram, such as functional residual capacity (FRC) and the content of carbon dioxide in the venous blood (CᵥCO₂) of a subject, may escape consideration, potentially leading to an inaccurate estimate of cardiac output.

Accordingly, there is a need for a process by which measurements of blood carbon dioxide content, pulmonary capillary blood flow, cardiac output, and other cardiac, cardiopulmonary, and respiratory parameters may be noninvasively and accurately determined.

### SUMMARY OF THE INVENTION

The present invention includes methods for noninvasively evaluating indicators of the cardiopulmonary performance of a subject, such as cardiac output, pulmonary capillary blood flow, and blood carbon dioxide content.

The inventive methods include obtaining data of comprising a respiratory signal, e.g., a carbon dioxide signal, an oxygen (O₂) signal, a nitrogen (N₂) signal, an SF₆ signal, a helium (He) signal, a signal corresponding to an amount of an anesthetic agent present in respiration, etc., of a subject and comparing data generated by an algorithmic lung model to the data of the respiratory signal. Hereinafter, the example of an expiratory carbon dioxide signal is used to refer to the measured respiratory signal. The variables that are input into the algorithmic lung model are adjusted, e.g., through one or more iterations, and calculations repeated, until the data generated thereby reflects that of the measured expiratory carbon dioxide signal with a desired degree of accuracy. Once the algorithmic lung model replicates the data of the measured expiratory carbon dioxide signal with the desired degree of accuracy, one or more of the input, or measured, values may be used to determine an indicator of the cardiopulmonary health or performance of the subject.

The present invention also includes computer programs for generating a multi-component mathematical lung model and for using such a model or portion thereof to noninvasively evaluating the cardiopulmonary health or performance of a subject. Computers and other processing elements that are configured to execute such programs are also within the scope of the present invention.

In addition, the present invention includes systems for noninvasively evaluating indicators of the cardiopulmonary health or performance of a subject. Such systems include apparatus for monitoring at least one gas in the respiration of the subject, as well as the flow of the subject's respiration, and at least one processing element for generating a multi-component mathematical model of the lung or lungs of a subject, or a portion thereof, for modifying the model to accurately resemble measured parameters, and for using the model to noninvasively evaluate one or more indicators of the cardiopulmonary health or performance of the subject.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is a schematic representation of a conventional rebreathing system;

FIG. 2 is a schematic representation of the conventional rebreathing system of FIG. 1, illustrating an exemplary deadspace in greater detail than that shown in FIG. 1;

FIG. 3A is a schematic representation of the airway and lungs of a subject;

FIG. 3B is a schematic representation of a multi-component mathematical model of the lung or lungs of a subject;

FIG. 3C is a schematic representation of various generations of a lung, including branching of airways and alveoli;

FIG. 4 is a schematic representation of a system incorporating teachings of the present invention;

FIG. 5 is a schematic representation depicting an exemplary method of using a multi-component mathematical lung model in accordance with teachings of the present invention; and

FIG. 6 is a graph depicting comparison of actual measured values with measured values that have been used to provide an accurate multi-component mathematical lung model of a subject being evaluated.

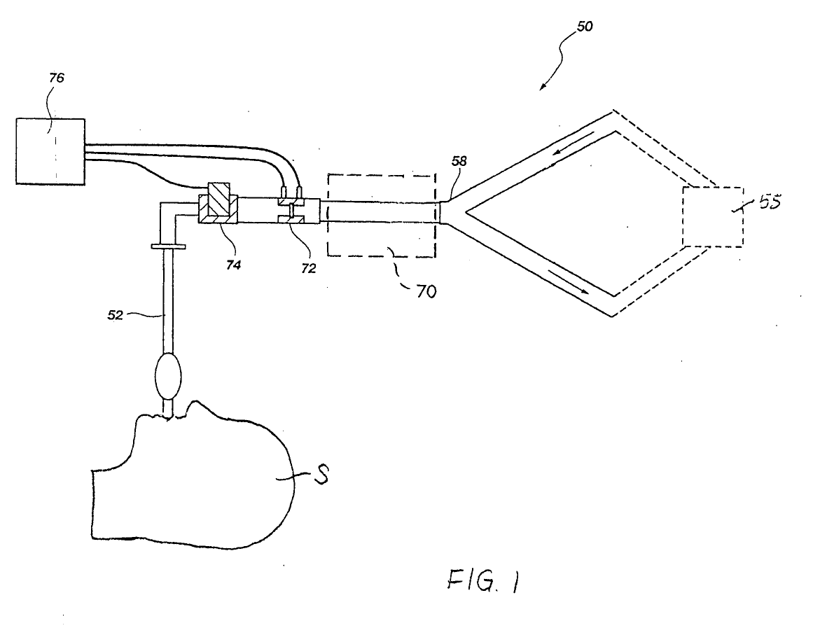

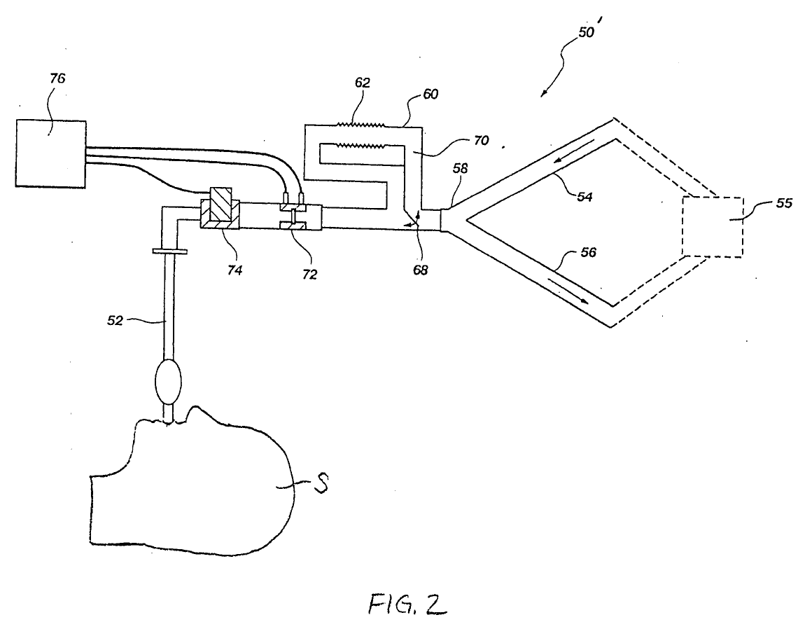

### DETAILED DESCRIPTION OF THE EXEMPLARY EMBODIMENTS

The present invention includes methods for noninvasively determining one or more indicators of the cardiopulmonary health of a subject. In the inventive methods, carbon dioxide and flow signals of a subject are monitored and the data therefrom evaluated over the course of a predetermined period of time, which exceeds the length of an entire single breath (inspiration and expiration). The carbon dioxide and flow data are then evaluated and compared with a multi-component mathematical model of the subject's lung, e.g., a human lung, the lung of another mammal, the lung of another animal, etc., into which values for one or more indicators of the cardiopulmonary health of the subject are input.

For example, an indicator such as cardiac output (or pulmonary capillary blood flow), functional residual capacity (FRC), or the content of carbon dioxide in the mixed venous blood of the subject (CᵥCO₂) may be input into the multi-component mathematical model. If the carbon dioxide and flow data generated by the multi-component mathematical model of the lung differ significantly, e.g., by more than a predetermined threshold percentage, from the carbon dioxide and flow data that has been collected, the values of one or more of the indicators may be adjusted, then the data regenerated. Once the regenerated carbon dioxide and flow data no longer differ significantly from the corresponding collected carbon dioxide and flow data, the input values of the indicators are considered to be optimized and may be identified and supplied to a user, such as a healthcare professional, to provide the user with an indication of the subject's cardiopulmonary health or performance.

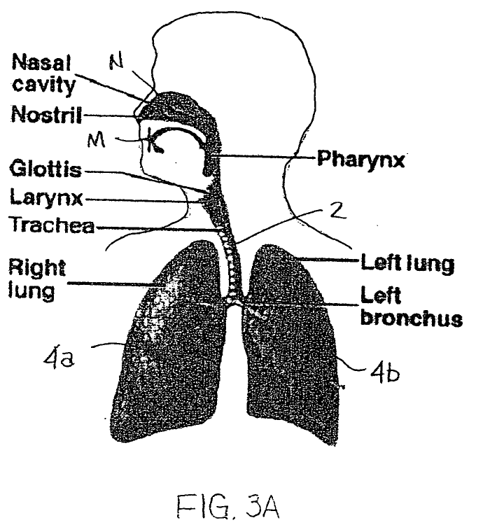

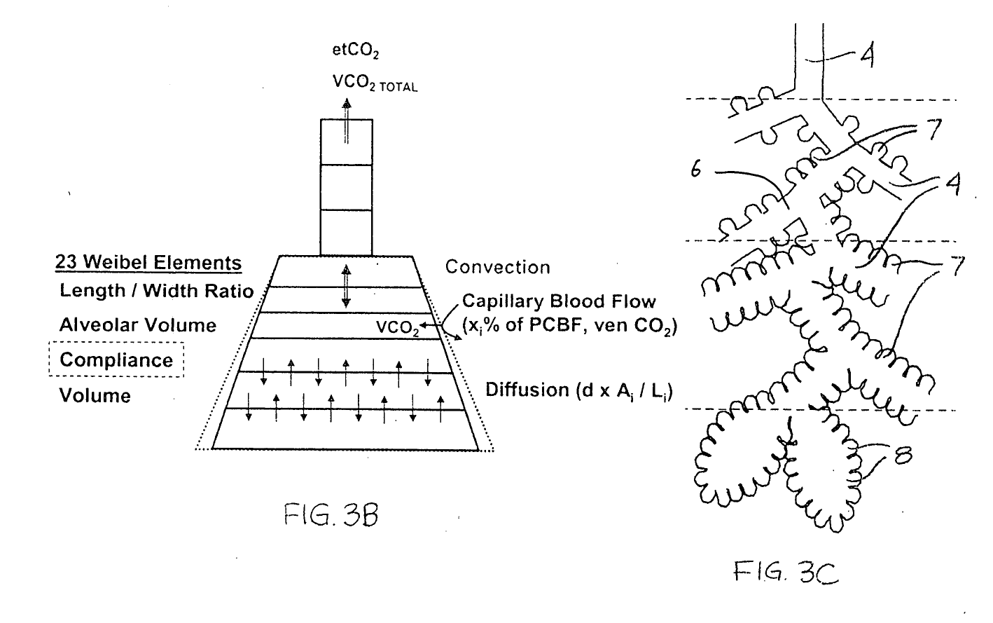

In one aspect, the present invention includes a multi-component mathematical model of the lung or lungs of a subject. By way of nonlimiting example, the multi-component mathematical model may be based on a human mouth M and nose N, a human trachea 2, and a pair of human lungs 4a and 4b, as are schematically depicted in FIG. 3A. As a more specific example, the multi-component mathematical model may be based on the so-called “Weibel model” of the human lungs or a variant thereof.

The multi-component mathematical model may account for a variety of lung features, including, without limitation, the anatomy or morphology of the modeled lung and manner in which gases flow through the modeled lung. In the present invention, a modified version of the Weibel model, which is referred to as the “Utah Weibel Model,” may be employed to provide a noninvasive measurement of one or more indicators of the cardiopulmonary health of a subject. As shown in FIG. 3B, the Utah Weibel model incorporates the detailed morphological descriptions of the lung, which were published in Weibel, ER, Morphometry of the Human Lung, 1st Ed., Chapter XI (Academic Press Publishers, 1963) (hereinafter “Weibel”), the disclosure of which is hereby incorporated herein in its entirety by this reference. In addition to considering the anatomical characteristics of the lung (length and width of airways, number of alveoli per airway, size of alveoli, etc.), as provided by the Weibel model, the Utah Weibel Model also accounts for various other aspects of the lung, including alveolar gas exchange and gas transport, e.g., diffusion, convection, etc.

In a multi-component mathematical model of the lung that incorporates teachings of the present invention, anatomical aspects are accounted for as a series of generations, which may begin with the trachea 2 (FIG. 3A), which is also referred to as the “0^{th} generation,” or upstreams thereof, e.g., at the breathing tube, a sensor, etc., and continuing on, with each successive generation of airway 6 branching, with some nonterminal alveoli 7 being present along various airways 6, to terminal alveoli 8, as shown in FIG. 3C. In the Weibel and Utah Weibel multi-component mathematical models, terminal alveoli 8 are also referred to as the “23^{rd} generation.”

The number of airways (m) in each generation (z) of the Weibel and Utah Weibel models is defined by the following equation:
m(z)=2^{z} (8)

The number of alveoli 7, 8 (N_{A}) along each airway 6 of the various generations (z) of the Weibel and Utah Weibel models is set forth in the following TABLE:

| | Number of Alveoli |
|---|---|
| Generation (z) | per Airway |
| 0-16 | 0 |
| 17 | 5 |
| 18 | 8 |
| 19 | 12 |
| 20 | 20 |
| 21 | 20 |
| 22 | 20 |
| 23 | 20 |

The total number of alveoli 7, 8, in each generation is defined by multiplying the number of airways 6 (m) in a generation (z) by the number alveoli 7, 8, per airway 6 in that generation (N_{A}(z)). The total number of alveoli 7, 8 (N_{T}) in the multi-component mathematical model of the lung (FIG. 3B) is determined by summing N_{A}(z) for each multi-component mathematical model of the lung.

In reality, a human lung may include as few as eight generations or more than twenty-four generations. Thus, multi-component mathematical lung models that differ from the Weibel models are also within the scope of the present invention.

Morphologically, each generation, or segment, of such a multi-component mathematical model of the lung is specified in terms of a particular number of airways and a particular number of alveoli. In addition, each generation of such a multi-component mathematical model of the lung is morphologically defined in terms of the dimensions (e.g., diameter of each airway, cross-sectional area of each airway, length of each airway) of the airways of that generation and, thus the total volume of that particular generation. In the Weibel and Utah Weibel models, these dimensions and volumes are based upon data published by Weibel, which was obtained from a detailed dissection of a human lung.

Because various lung models may be based on particular lungs, such as the Weibel model's dependence upon the characteristics of the human lung that was dissected by Weibel, it is well known in the art that, in order to obtain a lung model that represents the lungs of a subject more accurately than the basic Weibel model, the lung model may be scaled based on one or more of a variety of factors. Exemplary methods for scaling a multi-component mathematical lung model and, more specifically, for obtaining a more accurate estimate of the FRC of an individual, are described in Schwardt, J D, et al., “Sensitivity of CO₂ washout to changes in acinar structure in a single-path model of lung airways,” ANN. BIOMED. ENG. 19:679-97 (1991) (“Schwardt”).

In Schwardt, the scaling factor that corresponds to a particular generation of the lung (S(z)) is based on the volume of the FRC (V_{FRC}), measured in milliliters (mL), for that generation and is defined by the following equation:
S(z)=(V_{FRC}−V_{Weibel 0-4})/(V_{Weibel FRC}−V_{Weibel 0-4}), (9)
where V_{Weibel 0-4}, which represents the volume of each of the 0^{th} through fourth generations (z=0 through 4) of the pair of lungs of the Weibel model, is equal to 47.2946 mL, the FRC of the pair of lungs of the Weibel model (V_{Weibel FRC}) has a volume of 4800 mL (which is unevenly split between the various generations of the lung model), and V_{FRC} is the volume of the FRC of the subject whose lung is to be mathematically modeled for an evaluation of cardiopulmonary health. V_{FRC} may be an estimated value, as described in greater detail hereinafter, or a known value. S(z) accounts for the expansion of the alveoli during breathing. Such expansion, as is evident from the parameters used in calculating S(z), is at least partially a function of the area and length of the airways of the lung model. The scaling factor is used, along with various other parameters, in determining the volume of each generation of the multi-component mathematical lung model of the present invention.

In determining the volume of each generation (z) of the multi-component mathematical lung model, the cross-sectional area, length, and diameter of each of the airways in that generation are first determined. The cross-sectional area (A(z)) of the interface between two generations of the lung model, which cross-sectional area is measured in square centimeters (cm²), may be determined as follows for each of the ^{th} to fourth generations (i.e., between generations 0 and 1, 1 and 2, 2 and 3, 3 and 4) of the Weibel model of a pair of human lungs:
A(z)=β×2.54×e^{-0.083z}, (10)
where e has a known numerical value and, β is a factor which facilitates modeling of airways of different shapes (e.g., longer and thinner vs. shorter and wider). β, which is a value between zero and one, varies from subject to subject but remains fixed for a particular subject and, thus, remains constant throughout all of the formulas that are used to generate carbon dioxide and flow data in accordance with teachings of the present invention. β may be iteratively determined as the algorithms described herein are used to generate a multi-component mathematical lung model that accurately resembles all or part of a lung or pair of lungs of a subject undergoing evaluation. The values 2.54 and −0.083 are factors that are used to adjust A(z) to the correct units (cm²), as both β and e are dimensionless.

The cross-sectional area (A(z)) at the interfaces between generations for each of the fourth through twenty-third generations of the Weibel model of a pair of human lungs may be determined by the equation:
A(z)=S(z)^{(2/3)}×β×1.32×e^{0.1074z+0.0125 zˆ2} (11)
S(z), which is the scaling factor determined by equation (9) above, is included in equation (11) to account for expansion of the alveoli, which do not begin appearing in the lung or a lung model until about the fourth generation. As a result, the alveoli and expansion thereof are considered in determining the lengths and areas of the airways of each generation of the lung model. The values 1.32, 0.1074, and 0.0125 are factors that are used to adjust A(z) to the correct units (cm²), as both β and e are dimensionless.

The length (l(z)) of each airway within each of the 0^{th} through third generations of a particular generation of a multi-component mathematical lung model according to the present invention, which is measured in cm, may be determined by the following equation:
l(z)=(12.0×e^{-0.92z})/β. (12)

The values 12.0 and −0.92 are factors that are used to adjust l(z) to the correct units (cm), as both β and e are dimensionless. For the fourth through twenty-third generations, the following formula is useful for determining l(z):
l(z)=S(z)^{(1/3)}×(2.5×e^{-0.17z})/β (13)

The values 2.5 and −0.17 are factors that are used to adjust l(z) to the correct units (cm), as both β and e are dimensionless. Notably, l(z) for the 0^{th} through third generations does not account for alveoli, as alveoli are typically not present along the airways of these generations, while l(z) for the fourth through twenty-third generations does account for the alveoli that are typically present along such generations.

The average diameter of each airway of a particular generation (d(z)) of the multi-component mathematical lung model may be determined by way of the following equation:

$$d ( z ) = 2 \times \sqrt{\frac{A ( z )}{π \times m ( z )}} , \qquad ( 14 )$$

where π has a known numerical value, i.e., about 3.14, A(z) is determined in accordance with either equation (10) or equation (11), and m(z) is determined in accordance with equation (8). Notably, d(z) does not account for the presence of alveoli along the airways.

The total volume of all of the alveoli in each of the generations (V_{ALV Z}) of a multi-component mathematical lung model according to the present invention may initially be estimated, then calculated by the following equation:
V_{ALV}(Z)=N_{A}(Z)×V_{ALV TOTAL}/Σ(N_{A}(i), i=0 . . . 23), (15)
where V_{ALV TOTAL}, which is equal to the summation of the volumes of each of the alveoli (Vₐₗᵥ) in the lung, e.g., Σ(Vₐₗᵥ(i), i=0 . . . 23, is the total alveolar volume of all alveoli in the whole lung, N_{A}(z) is the number of alveoli in a particular generation of the lungs and may be determined as indicated previously herein, and N_{A}(i), i=0 . . . 23, represents the number of alveoli in all generations of the multi-component mathematical lung model.

As ventilation of the alveoli of a subject may not be homogeneous, equation (15) may be modified to account for a percentage or fraction of alveoli that are only partially ventilated or that are not at all ventilated. In addition to accounting for the morphology, or anatomical dimensions of a multi-component mathematical lung model, an accounting of the manner in which gases move through such a lung model may also be made. Several mechanisms may be responsible for gas movement through a lung, including, but not limited to, simple mass transport, i.e., convection, diffusion between adjacent generations, or segments, and diffusion between the gas phase and the blood phase, e.g., at the gas-blood interface of each alveolus.

In modeling the mass transport, or convection, of gases into and out of the lung, the volume and concentrations of gases within each generation of a multi-component mathematical model of the lung are evaluated each time respiratory flow and carbon dioxide signals are sampled (n). The interval between sample times (n) is represented as Tₛₐₘₚₗₑ. By way of example only, samples may be obtained at frequency of about ten times each second to about one hundred times each second, with about fifty times per second being typical in conventional respiratory monitoring processes. Mass transport is modeled in an incremental basis for each sample time (n), starting with the generation (z) of the multi-component mathematical model of the lung in which gas flow is initiated. For example, during inspiration, mass transport is modeled at the mouth and trachea (z=0), then at each successive generation (z=1, 2, 3, . . . ), until mass flow into the final generation of the multi-component mathematical model of the lung (e.g., z=23) is modeled. Conversely, when mass transport is evaluated during expiration, the deepest generation (e.g., z=23) of the multi-component mathematical model of the lung is first modeled, then each less complex generation is modeled until, finally, mass transport of gases out of the 0^{th} generation is modeled.

Knowing various characteristics of a multi-component mathematical model of the lung, such as those described above, is useful in noninvasively evaluating one or more cardiac, respiratory, or cardiopulmonary parameters of a subject.

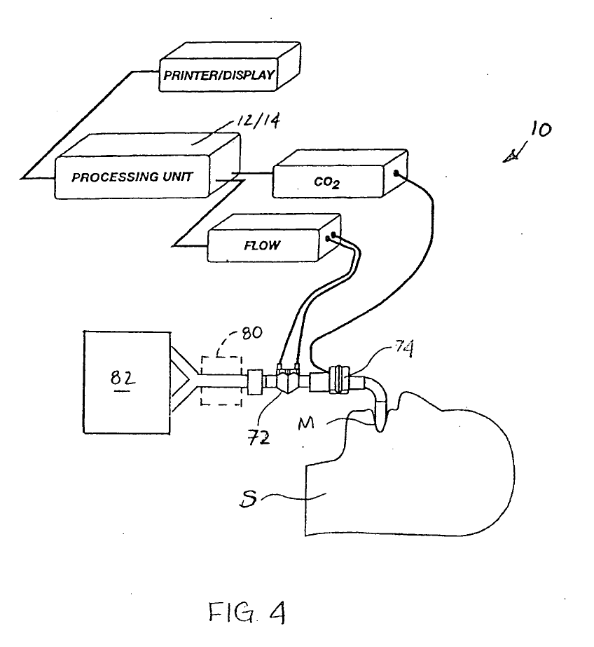

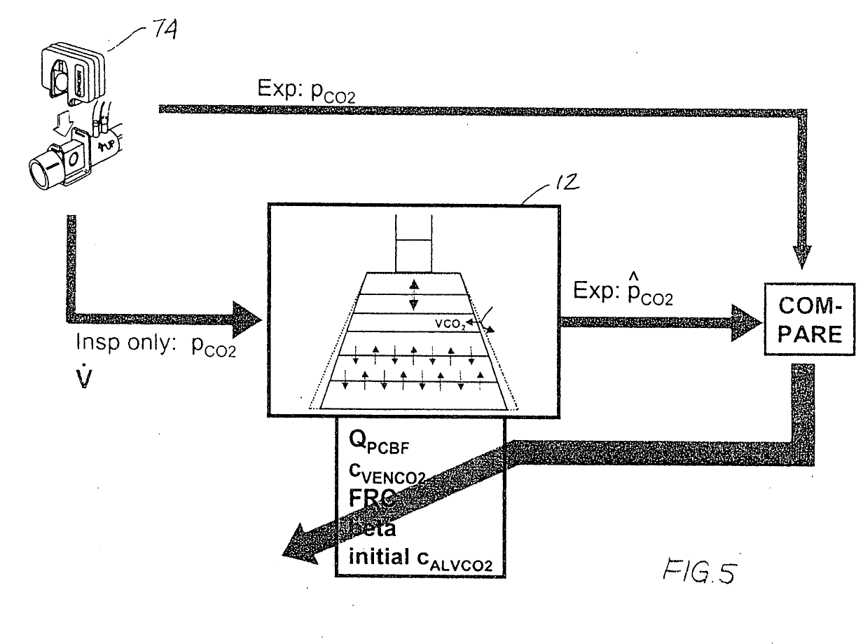

At the mouth M of the subject S, mass transport may be measured with a flow meter 72 of a known type, as shown in FIG. 4, such as one of the differential pressure tachometers available from Respironics, Inc., of Murrysville, Pa. For example, flow meter 72 may be combined with a CAPNOSTAT® carbon dioxide sensor 74 and rebreathing valve part as part of the disposable NICO® sensor model no. 8950, which may be used in conjunction with the NICO₂® model 7300 monitor, also available from Respironics, Inc.

A sample of the flow signal (Flow(n)) at a particular sample time (n) may be multiplied by the amount of time between samples (e.g., n−1, n) (Tₛₐₘₚₗₑ) to determine the total volume of gas (ΔV_{T}(n)) that is drawn into mouth M of subject S over a time period that is equal to Tₛₐₘₚₗₑ and during which the sample was obtained:
ΔV_{T}(n)=Flow(n)×T_{Sample} (16)

Once the total volume of gas (ΔV_{T}(n)) that is drawn into mouth M at a time interval that corresponds to sample time (n) has been determined, the volume of gas that flows into each subsequent generation (z) (ΔV_{IN}(n,z)) over the same duration of sample time (n) may also be determined, such as by use of the following equation:
ΔV_{IN}(n,z)=ΔV_{T}(n)×{1−1/N_{T}×Σ(N_{A}(i), i=0 . . . z)} (17)

Again, N_{A} represents the number of alveoli in the evaluated generation (z) and N_{T} represents all of the alveoli that are present in the multi-component mathematical model of the lung. Equation (17) is based on the negligible ability of the airways of a lung or pair of lungs to expand and, thus, to take up a volume of gases that exceeds the capacity or capacities thereof, but recognizes the ability of alveoli to expand and, thus, to take up a volume of gases which exceeds their relaxed volumes. In particular, the {1−1/N_{T}×(N_{A}(i), i=0 . . . z)} section of equation (17) accounts for the expandability of each alveolus that is present in the evaluated generation (z) of the multi-component mathematical model of the lung, as well as every alveolus in all of the preceding generations (i.e., generations 0 through z−1; those through which at least some of the sampled gases have already theoretically flowed). As can be seen from this portion of equation (17), it is assumed that substantially the same volume of gases that is inhaled at the mouth at a particular sample time (n) flows through each generation of the multi-component mathematical model of the lung that lacks alveoli (i.e., if Σ(N_{A}(i), i=0 . . .z) is equal to zero, ΔV_{IN}(n,z) is assumed to be substantially equal to ΔV_{T}(n)).

When the volume of gases that has flowed into a particular generation of the multi-component mathematical model of the lung as a result of flow measured at a specific sampling time (n) has been calculated, the concentration of carbon dioxide in such gases may also be determined. Such a determination may be made based on a carbon dioxide signal that was sampled at sampling time (n), as well as upon the total volume of the airways and alveoli in the generation (z) of the multi-component mathematical model of the lung which is being evaluated, as known in the art. With reference again to FIG. 4, a carbon dioxide signal that represents the gases present at mouth M of subject S, or the 0^{th} generation of the multi-component mathematical model of the lung, may be obtained at each sample time (n) by use of a carbon dioxide sensor 74, or capnometer, of a type known in the art, such as one of the capnometers available from Respironics, Inc. Carbon dioxide sensor 74 may be combined with or separate from flow meter 72. For example, carbon dioxide sensor 74 may a CAPNOSTAT® sensor, which may be combined with a flow meter and rebreathing valve in the disposable NICO® sensor model no. 8950, available from Respironics, Inc.

Alternatively, an oxygen sensor of known type may be used in place of a capnometer. Of course, when an oxygen sensor is used, oxygen uptake or consumption (VO₂), rather than carbon dioxide production or elimination, is measured. Other substances (e.g., gases) that may be measured include, without limitation, N₂, SF₆, He, anesthetic agents, and the like. Known uptake, or absorption, or consumption formulas may be sued to track the flow of such substances through a multi-component mathematical model of the lung in accordance with teachings of the present invention, as appropriate for the substance being monitored.

Since gases are already contained within each generation (z), i.e., lungs typically do not totally collapse during exhalation, as more gases flow into a generation (z) of the lungs, some gas mixing occurs. Taking these factors into consideration, the volume of carbon dioxide that flows into a generation (z) of a multi-component mathematical model of the lung which incorporates teachings of the present invention as the result of respiration measured at a specified sampling time (n) (ΔV_{CO2}(n,z)) may be estimated by the following equation:
ΔV_{CO2}(n,z)=ΔV_{IN}(n,z)×conc_{CO2}(z−1,n), (18)
where ΔV_{IN}(n,z) is the total volume of gases that theoretically flow into generation (z), as calculated by use of equation (17), and conc_{CO2}(z−1,n) is the concentration of carbon dioxide in the gases of the previous generation (z−1) through which the gases that were evaluated at sample time (n) theoretically flowed before reaching generation (z). At the 0^{th} generation, conc_{CO2}(z−1,n) is the concentration of carbon dioxide at the mouth M of subject S, as determined by known techniques, such as from the carbon dioxide signal that generated by carbon dioxide sensor 74 or the oxygen signal that is generated by an oxygen sensor. For example, at mouth M, conc_{CO}₂(z−1, n) is equal to the measured partial pressure of carbon dioxide (pCO₂) divided by the measured barometric pressure (p_{bar}). In subsequent generations, concCO₂(z−1, n) is affected by convection and, thus, may be determined, in view of the estimated volume within each generation of a multi-component model of the lung of subject S, by estimating the diffusion of carbon dioxide (or, alternatively, oxygen) between the blood and respiratory gases between different generations of the multi-component model of the lung.

As subject S inspires, carbon dioxide within a particular generation (z−1) will be drawn into the next successive generation (z), thereby changing the volume of carbon dioxide present within that next successive generation (z). In addition, as gases flow into a generation (z) from the previous generation (z−1), mixing occurs as gases that were already within the evaluated generation (z) are displaced into the next, successive generation. Such mixing may be accounted for by the following equation, which considers both the volume of carbon dioxide flowing into the evaluated generation (z) (ΔV_{CO2}(n,z)), as determined by use of equation (18), as well as the volume of carbon dioxide that was theoretically present in the evaluated generation (z) during the previous sampling time (n−1) (V_{CO2}(n−1,z)), as was estimated in accordance with equation (18) for the previous sampling time (n−1):
V_{CO2}(n,z)=V_{CO2}(n−1,z)+ΔV_{CO2}(n,z). (19)

An initial value for V_{CO2}(n−1,z) may be made based on measurement of the partial pressure of end tidal carbon dioxide (PetCO₂) during the immediately preceding expiratory phase of subject S's breathing, as well as on the simultaneously sampled respiratory flow. Of course, as an alternative to estimating V_{CO2} at each generation of the lung model, V_{O2} may be monitored and estimated in a similar fashion.

Equation (19) is based on the assumption that instant and complete mixing occurs within each generation (z) of a multi-component mathematical lung model, an assumption that does not always hold. For example, isolated portions of one generation (z) communicate with isolated portions of the preceding and ensuing generations, (z−1) and (z+1), respectively, through individual airways (e.g., the left and right bronchus), between which mixing is not likely to occur instantaneously or to be complete. Accordingly, variations of equation (19), as well as other algorithms that take such noninstantaneous and incomplete mixing into consideration, are also within the scope of the present invention.

The volume of carbon dioxide that flows into the preceding generation during sample time (n) may be determined by considering ΔV_{CO2}(n,z) and the volume of carbon dioxide that is theoretically present in the preceding generation (z−1) at sampling time (n), as determined by equation (18), in accordance with the following equation:
V_{CO2}(n,z−1)=V_{CO2}(n,z−1)−ΔV_{CO2}(n,z). (20)

An initial determination of V_{CO2}(n,z−1), prior to the 0^{th} generation, may be made based on the respiratory flow and carbon dioxide signals that are measured by carbon dioxide sensor 74 at mouth M of subject S. V_{CO2}(n, z−1) is updated for each generation of the lung model and, thus, recalculated with each subsequently evaluated generation (z).

The volume of gases within the alveoli of a particular generation (z) of the multi-component mathematical model of the lung at a particular sample time (n) (V_{ALV}(n,z)) may be estimated with the following equation:
V_{ALV}(n,z)=V_{ALV}(n−1,z)+ΔV_{IN}(n,z), (21)
where ΔV_{IN}(n,z) is determined in accordance with equation (17) and V_{ALV}(n−1,z) is initially an estimated alveolar volume for generation (z), which is a portion of an overall estimated alveolar volume, then becomes a calculated value as iterations occur and algorithms of the present invention are recalculated. By way of example only, V_{ALV}(n−1,z) may be estimated to be a portion of a total estimated alveolar volume of 3,600 mL or an alveolar volume (total or specific to generation (z)) that is based upon the most recently evaluated set of data that corresponds to subject S. As ventilation of the alveoli of a subject may not be homogeneous, V_{ALV}(n−1,z) may be modified to account for a percentage or fraction of alveoli that are only partially ventilated or that are not at all ventilated.

The volume of gases within the alveoli of the previous generation (z−1) from which gases are drawn during a specific sample time (n) may be determined as follows:
V_{ALV}(n,z−1)=V_{ALV}(n−1,z−1)−ΔV_{IN}(n,z), (22)
where ΔV_{IN}(n,z) is determined in accordance with equation (17) and V_{ALV}(n−1,z−1) is initially a set value (e.g., zero, as the generations 0-3 are typically deemed to lack alveoli) and may subsequently be the value determined by use of equation (21 ) above for a previous generation. Since ventilation of the alveoli of a subject may not be homogeneous, equation (22) may be modified to account for a percentage or fraction of alveoli that are only partially ventilated or that are not at all ventilated.

As the direction of air flow during expiration is opposite that in inspiration, the equations that follow are updated in the reverse order than that in which the equations that correspond to inspiration are updated; from the deepest generation (e.g., the 23^{rd} generation) to the shallowest generation (e.g., the 0^{th} generation) for each sample time (n).

The total volume of gas that moves into a generation (z) at a particular sample time (n) (ΔV_{T}(n)) is a function of the total flow (Flow(n)) out of the 0^{th} generation during the sampling time (n):

ΔV_{T}(n)=Flow(n)×Tₛₐₘₚₗₑ. (23)

Once ΔV_{T}(n) has been determined, assuming that the airways do not expand significantly and that the only structures of the multi-component mathematical model of the lung that give up volume are the alveoli, the volume of gases that flows out of a particular generation (z) of the multi-component mathematical model of the lung during sample time (n) (ΔV_{OUT}(n,z)) may be estimated as follows:
ΔV_{OUT}(n,z)=ΔV_{T}(n)×1/N_{T}×Σ(N_{A}(i), i=z . . . 23), (24)
where N_{A} represents the number of alveoli in each generation from the deepest (e.g., z=23) to that being evaluated (see TABLE, above), N_{T} represents the total number of alveoli in the multi-component mathematical model of the lung, and ΔV_{T}(n) is the value determined by use of equation (23).

When ΔV_{OUT}(n,z) has been estimated, the volume of carbon dioxide flowing into the evaluated generation (z) at sample time (n) (ΔV_{CO2}(n,z)) may also be estimated:
ΔV_{CO2}(n,z)=ΔV_{OUT}(n,z)×conc_{CO2}(z+1,n), (25)
where conc _{CO2}(z+1,n) is the concentration of carbon dioxide flowing into the evaluated generation (z) from the successive generation (z+1) (e.g., from the 23^{rd} generation into the 22^{nd} generation).

With these definitions, the total volume of CO₂ (V_{CO2}) in each generation (z and z+1) during sample time (n) may be updated as follows:
V_{CO2}(n,z)=V_{CO2}(n−1,z)+ΔV_{CO2}(n,z) (26)
_{CO2}(n,z+1)=V_{CO2}(n,z+1)−ΔV_{CO2}(n,z) (27)

The volume of gases residing within the alveoli (V_{ALV}) of each generation (z and z+1) at a particular sample time (n) may also be determined, as follows:
V_{ALV}(n,z)=V_{ALV}(n−1,z)+ΔV_{IN}(n,z), (28)
V_{ALV}(n,z+1)=V_{ALV}(n−1,z+1)−ΔV_{IN}(n,z) (29)

After convection-based calculations of the volume of gases within the alveoli (V_{ALV}) of each generation of the multi-component mathematical model of the lung and the volume of carbon dioxide (V_{CO2}) in each generation have been made, these values may be modified by considering one or both of the diffusion of gases, including carbon dioxide, between the gas and blood phases, as well as the diffusion of gases, including carbon dioxide, between each generation (z) of the multi-component mathematical model of the lung.

Diffusion of gases between the gas phase and the blood phase and, more particularly, of carbon dioxide from the blood into the lungs, may be modeled in accordance with the Fick principle. In general, the portion of the Fick equation that happens at a particular generation (z) of the multi-component mathematical model of the lung, or the amount of carbon dioxide that diffuses into the gases within the alveoli (ΔV_{CO2BLOOD}) of a particular generation (z) of the multi-component mathematical model of the lung at a specific sample time (n) is proportional to the number of alveoli in that generation (z), and may be modeled by the following equation:
ΔV_{CO2BLOOD}(n,z)=Q_{PCBF}×(N_{A}(z)/N_{T})×{content_{CO2VEN}(n)−content(conc_{CO2}(n,z))}, (30)
where Q_{PCBF} represents total pulmonary capillary blood flow, a value which is estimated and adjusted in accordance with teachings of the present invention, N_{A} is the total number of alveoli in the evaluated generation, N_{T} is the total number of alveoli in the multi-component mathematical model of the lung being employed, content(conc_{CO2}(n,z)) is a content value which is determined by use of a standard carbon dioxide dissociation curve, and content_{CO2VEN}(n) (or CvCO₂) is the content of carbon dioxide in the venous blood of subject S at sample time (n). The value for content_{CO2VEN} (n) is an estimated value (based on barometric pressure (P_{bar}), VCO₂, and a standard carbon dioxide dissociation curve) this is to be iteratively optimized along with cardiac output or PCBF, FRC, the initial content of carbon dioxide in the alveoli (CACO₂), and β, which may be assumed to remain constant or to vary (e.g., by modeling recirculation of blood), and which may be adjusted in accordance with teachings of the present invention. As N_{A} and N_{T} are respectively the number of alveoli in an evaluated generation (z) of the lung model and the total number of alveoli in the lung model being used, N_{A}(z)/N_{T} represents the percentage of the total model to which the calculated diffusion applies.

Equation (30) is based on the assumption that the blood flow is the same in all of the alveoli of the multi-component mathematical lung model. As it is well known that this assumption does not always hold, equation (30) may be modified so that the distribution of blood flow through the multi-component mathematical lung model does depend upon the total number of alveoli in the lung. For example, it may be assumed that a certain percentage or fraction of the alveoli in one or more generations (z) of the multi-component mathematical lung model receive no blood flow or reduced blood flow.

Once ΔV_{CO2BLOOD}(n,z) has been estimated, V_{CO2}(n,z) may also be updated, such as with the following equation:
V_{CO2}(n,z)(updated)=V_{CO2}(n,z)(prior)+ΔV_{CO2BLOOD}(n,z), (31)
where V_{CO2}(n,z) (prior) is, for example, the value obtained from the calculation made with one or more of equations (19), (25), and (26) above. Alternatively, V_{CO2}(n,z) (prior) may also account for diffusion between adjacent generations of the multi-component mathematical model of the lung.

An accounting may also be made in the multi-component mathematical model of the lung for gas diffusion between different generations (z) of the multi-component mathematical model. Such an accounting may be based, for example, on Fick's law of diffusion. Diffusive gas movement is dependent on the concentration difference and the area between the generations (z) of the multi-component mathematical model of the lung, as well as the length over which such diffusion occurs and the area in which diffusion occurs over a given period of time, or “diffusivity” (D).

At each sample time (n), the volume of carbon dioxide that diffuses into a generation (z) from the previous (i.e., upstream during inspiration) generation (z−1) (ΔV_{CO2DIFFUSION}(n,z)) is updated from the 0^{th} generation to the deepest generation (e.g., the 23^{rd} generation), as follows:
ΔV_{CO2DIFFUSION}(n,z)=D×A(z−1)/avg{l(z), l(z−1)}×{conc_{CO2}(n,z−1)−conc_{CO2}(n,z)} (32)
where D is a specified value, such as 0.17 cm²/s, A(z−1) is the cross-sectional area of each airway in generation (z−1), as may be determined in accordance with equation (10) or equation (11) above, l(z) and l(z−1) are the lengths of each airway in generation (z) and generation (z−1), as modeled by one or both of equations (12) and (13) above, and conc_{CO2}(n,z−1) and conc_{CO2}(n,z) are the modeled concentrations of carbon dioxide in generations (z) and (z−1) at sample time (n), as estimated (e.g., from previous calculations, based on one or more characteristics of subject S, etc.).

Upon determining the volume of carbon dioxide that diffuses from one generation (z−1) into the next (z), the volumes of carbon dioxide that are within each generation (z−1 and z) at sample time (n) may be updated once per sample time (n) to account for diffusion therebetween, as follows:
V_{CO2}(n,z)(updated)=V_{CO2}(n,z)(prior)+ΔV_{CO2DIFFUSION}(n,z), (33)
V_{CO2}(n,z−1)(updated)=V_{CO2}(n,z−1)(prior)−ΔV_{CO2DIFFUSION}(n,z), (34)
where V_{CO2}(n,z) (prior) and V_{CO2}(n,z−1) (prior) are, for example, the values obtained from the calculation made with any of equations (18) through (20), (25) through (27), or (31) above.

An example of use of a multi-component mathematical model of the lung such as that set forth in equations (8) through (34) includes, for a particular sample time (n), determining the convection that occurs, as well as the diffusion of carbon dioxide between blood and gas. For each sample time (n), the preceding equations are combined and substantially continuously recalculated (e.g., by modifying PCBF, CvCO₂, β, initial CACO₂, etc.) to update each generation (z), or segment, of the multi-component mathematical model of the lung until each generation (z) of the lung model has been updated at least once during that sample time (n) and until the calculations that are based upon the multi-component mathematical model of the lung approximate measured values obtained during expiration with flow meter 72 and carbon dioxide sensor 74 within a desired degree of accuracy:

if inspiration for(z=0 . . . 23) {InspirationConvectionUpdate(z)}, else if expiration:

for(z=23 . . . 0){ExpirationConvectionUpdate(z)}; and

for(z=0 . . . 23){DiffusionFromBloodUpdate(z)DiffusionUpdate(z)}.

Although the present invention has been described in terms of a lung model which includes twenty-four generations (i.e., generations 0-23), lung models with other numbers of generations, as well as generations in addition to those of the lungs, are also within the scope of the present invention. By way of nonlimiting example, the 0^{th} generation of the multi-component mathematical model of the lung described in detail herein may comprise an endotracheal tube and additional generations of the lung model may include a filter (generation −1), a hose section (generation −2), etc, down to the location of a breathing circuit to which a gas sensor, e.g., a capnometer, oxygen sensor, anesthetic agent sensor, etc., is attached. Alternative apparatus and techniques may be used to conduct gas from a subject to flow and gas sensors, such as face masks or mouthpieces. These apparatus could likewise be incorporated into a lung model, which would take into consideration one or more of the configurations, volumes, and geometries of such apparatus. Additional generations may be beneficial in that the volumes thereof may be readily determined and the amounts of carbon dioxide or oxygen therein may be more directly determined than corresponding amounts within the lungs of a subject, providing more finite initial values when using a lung model according to the present invention.

A lung model of the present invention may also recognize and account for variations in resistance or compliance between different areas of the lung or lungs of a subject, e.g., areas of the lung in which flow occurs “fast” or “slow”. A lung model incorporating teachings of the present invention may likewise be based on a portion of one or more lungs of a subject.

Once the parameters of a multi-component mathematical lung model of the present invention have been optimized, the model is capable of estimating or predicting expiratory capnographic or other, e.g., O₂, N₂, SF₆, He, anesthetic agent, etc., waveforms at the mouth. Comparison of these estimated or predicted expiratory capnographic or other waveforms with actually measured waveforms facilitates optimization of the parameters of the multi-component mathematical lung model through a variety of methods, including gradient descent, iterative methods, random search, informed random search, and the like. Once a multi-component mathematical lung model has been optimized, it may be used to accurately estimate or predict values that may be used, as known in the art, to obtain accurate, noninvasive measurements of one or more of cardiac output, pulmonary capillary blood flow, and other cardiopulmonary parameters.

The teachings provided herein may also be applied to use of a multi-component mathematical lung model to predict continuous alveolar CO₂ or other, e.g., O₂, N₂, SF₆, He, anesthetic agent, etc., waveforms at the alveolar level.

Returning reference to FIG. 4, another aspect of the present invention is depicted. More specifically, a system 10 for noninvasively evaluating the cardiopulmonary health or performance of a subject is shown. In employing the system, a lung model according to the present invention may be used. The lung model may be generated electronically by one or more processors or other processing elements 12 under control of a computer program or group of programs, which may be stored in computer memory 14 or on one or more other storage media. The system and lung model may be used under substantially steady-state conditions (e.g., normal breathing) or under a variety of breathing conditions.

An exemplary computer program or programs that incorporate teachings of the present invention may include at least one collection element for collecting measured data corresponding to an amount of at least substance in respiration of a subject, e.g., CO₂, O₂, N₂, SF₆, He, an anesthetic agent, etc., and respiratory flow one over a period of more than one breath, e.g., from flow meter 72 and/or carbon dioxide sensor 74 or one or more flow and/or carbon dioxide monitors associated therewith. A generation element of a computer program of programs of the present invention may use the measured data to generate a multi-component mathematical lung model in accordance with teachings of the present invention. A comparison element of such a computer program or programs compares generated data from the multi-component mathematical lung model to the measured data. Such a comparison may include the comparison of one or more measured waveforms to one or more corresponding waveforms that are generated based on the multi-component mathematical lung model.

Once the measured and generated data have been compared, an adjustment element of the computer program or programs may adjust at least one value input into the multi-component mathematical lung model, e.g., FRC, cardiac output, PCBF, CvCO₂, β, initial CACO₂, etc. The comparison element may be configured to repeat comparison of the generated data to the measured data following adjustment of the at least one value input into the multi-component mathematical lung model until a minimum of a measure of the overall difference between the generated data and the measured data has been found.

The computer program or programs may also include an output element, which may, among other things, display, print, or otherwise output data of at least one indicator of the cardiopulmonary health of the subject, as obtained from the lung model. The output element may additionally cause one or more processors on which the computer program or programs are being executed to communicate with and, optionally, control operation of other elements of a system that includes the one or more processors, such as a perturbation (e.g., rebreathing) apparatus, a ventilator, or the like.

Initial data, which are also referred to herein as “starting values,” for one or more cardiopulmonary parameters may be entered into processing element 12 prior to generating a lung model according to the present invention. By way of example only, measured or estimated values for one or more of FRC, cardiac output, PCBF, CvCO₂, initial CACO₂, or the like may be entered into processing element 12. Entry of such data may increase the accuracy of the data obtained by generating a lung model, increase the speed with which the multi-component mathematical lung model is generated, or increase the rate at which one or more indicators of the cardiopulmonary health of a subject may be accurately determined.

As an example of use of the system and lung model under a variety of a breathing conditions, the breathing of subject S may be perturbated, e.g., by controlling a perturbation apparatus of a known type, such as a rebreathing apparatus 80 or a ventilator 82, to increase the amount of carbon dioxide in gases breathed by subject S, such as by inducing rebreathing, increasing or decreasing the rate at which subject S breathes, increasing or decreasing the volume of gases inhaled and exhaled by subject S, varying the duration, or pause time, between ventilator-induced breaths, etc., over the course of several breaths, e.g., for a duration of less than a minute, one minute, two minutes, three minutes, five minutes, or longer.

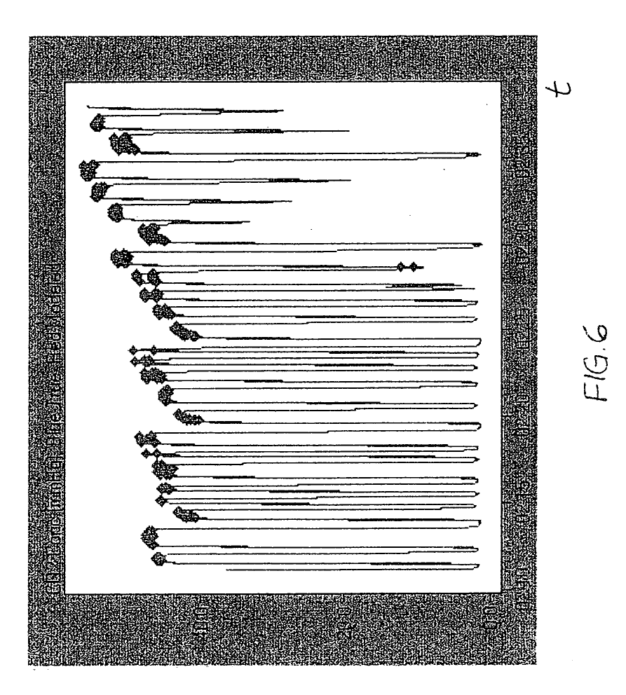

Once data has been collected, e.g., with flow meter 72 and/or carbon dioxide 74 sensor or one or more other suitable sensors, for a portion of a breath or for one or more breaths, the equations described herein or equivalent algorithms may be used to generate a multi-component mathematical model of the lung or lungs, or portion thereof, of a subject under evaluation, as shown by the schematic representation of FIG. 5 and the graph of FIG. 6. If the measured data and that generated with the multi-component mathematical lung model are not within a desired degree of accuracy, such as until a minimum of a measure, e.g., mean square sum, of the overall difference between generated and measured data has been found, the calculations may be repeated, until the desired degree of accuracy is achieved.

Once the desired degree of accuracy is achieved, data corresponding to various characteristics of the multi-component mathematical lung model, e.g., FRC, cardiac output, PCBF, CvCO₂, β, initial CACO₂, etc., or indicators of cardiopulmonary health, may be accurately and directly estimated. Some filtering, as known in the art, may be conducted on the estimated data to compensate for any “noise.”

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A method for determining an indicator of cardiopulmonary health of a subject, comprising:
obtaining measured data comprising signals corresponding to an amount of a substance in respiration and respiratory flow of the subject over more than one breath;
comparing generated data from an algorithmic lung model to the measured data;
adjusting at least one value input into the algorithmic lung model;
repeating the comparing at least once following the adjusting until a minimum of a measure of the overall difference between the generated data and the measured data has been found; and
following the repeating, identifying the indicator of cardiopulmonary health used in the algorithmic lung model.

2. The method of claim 1, wherein adjusting comprises adjusting a functional residual capacity (FRC), a cardiac output (Q or Q_{pcbf}), a content of carbon dioxide in venous blood (CᵥCO₂), or any combination thereof input into the algorithmic lung model.

3. The method of claim 2, wherein identifying comprises identifying a functional residual capacity (FRC), a cardiac output (Q or Q_{pcbf}), a content of carbon dioxide in venous blood (CᵥCO₂), or any combination thereof input into the algorithmic lung model.

4. The method of claim 1, wherein obtaining measured data comprises obtaining measured data comprising signals corresponding to carbon dioxide, oxygen, nitrogen, SF₆, helium, an anesthetic agent, or any combination thereof in a respiration gas of the subject.

5. The method of claim 1, wherein comparing, adjusting, and repeating comprise estimating a waveform of the substance.

6. The method of claim 1, wherein adjusting comprises employing a gradient descent technique, an iterative technique, a random search technique, an informed random search technique, or any combination thereof.

7. The method of claim 1, further comprising causing at least one perturbation in a respiration of the subject during the obtaining step by causing the subject to breathe gas mixtures including different amounts of the substance during the obtaining step, causing a respiratory rate of the subject to change during the obtaining step, causing a respiratory volume of the subject to change during the obtaining step, or any combination thereof.

8. A system for determining an indicator of cardiopulmonary health of a subject, comprising:
(a) a gas or vapor sensor for obtaining measured data comprising signals corresponding to an amount of a substance in respiration of the subject;
(b) a flow sensor for obtaining measured data comprising signals corresponding to a respiratory flow of the subject; and
(c) a processor programmed to:
(1) collect the measured data over more than one breath,
(2) compare generated data from an algorithmic lung model to the measured data,
(3) adjust at least one value input into the algorithmic lung model,
(4) repeat the comparison of the generated data to the measured at least once following adjustment the value until a minimum of a measure of the overall difference between the generated data and the measured data has been found, and
(5) following repetition of the comparison, identify the indicator of cardiopulmonary health used in the algorithmic lung model.

9. The system of claim 8, wherein the processor is programmed to adjust a functional residual capacity (FRC), a cardiac output (Q), a pulmonary capillary blood flow (Q_{pcbf}), a content of carbon dioxide in venous blood (CᵥCO₂), or any combination thereof input into the algorithmic lung model.

10. The system of claim 8, wherein the processor is configured to identifying an indicator of cardiopulmonary health comprising a functional residual capacity (FRC), a cardiac output (Q), a pulmonary capillary blood flow (Q_{pcbf}), a content of carbon dioxide in venous blood (CᵥCO₂), or any combination thereof input into the algorithmic lung model.

11. The system of claim 8, wherein the gas or vapor sensor is configured to sense oxygen, carbon dioxide, nitrogen, SF₆, helium, an anesthetic agent, or any combination thereof in respiration of the subject.

12. The system of claim 8, wherein the processor is programmed to estimate a waveform of the substance.

13. The system of claim 8, wherein the processor is programmed to employ a gradient descent technique, an iterative technique, a random search technique, an informed random search technique, or any combination thereof to adjust the value input into the algorithmic lung model.

14. The system of claim 8, wherein the processor is further programmed to directly or indirectly cause a respiration of the subject to be perturbated at least once while the measured data is being obtained.

15. The system of claim 8, further comprising a ventilator in communication with the processor, and wherein the processor is programmed to cause the ventilator to:
cause the subject to breathe gas mixtures including different amounts of the substance as the measured data is being obtained;
cause a respiratory rate of the subject to change as the measured data is being obtained;
cause a respiratory volume of the subject to change as the measured data is being obtained; or
vary pause times between ventilator-induced breaths.

16. A storage medium upon which a computer program is stored, the computer program being configured to be executed by a processor and comprising:
a collection element for collecting measured data corresponding to an amount of a substance in respiration gas of a subject and a respiratory flow one over a period of more than one breath;
a generation element for generating an algorithmic lung model based on the measured data;
a comparison element for comparing generated data from the algorithmic lung model to the measured data; and
an adjustment element for adjusting a value input into the algorithmic lung model, the comparison element being configured to repeat a comparison of the generated data to the measured data following adjustment of the value input into the algorithmic lung model until a minimum of a measure of the overall difference between the generated data and the measured data has been found.

17. The storage medium of claim 16, wherein the computer program further comprises an output element for outputting an indicator of cardiopulmonary health used in the algorithmic lung model once the collection, generation, comparison, and adjustment elements is complete.

18. The storage medium of claim 17, wherein the output element of the computer program is configured to output an indicator of cardiopulmonary health comprising at least one of the functional residual capacity (FRC), the cardiac output (Q), the pulmonary capillary blood flow (Q_{pcbf}), and the content of carbon dioxide in venous blood (CᵥCO₂) input into the algorithmic lung model.

19. The storage medium of claim 16, wherein the adjustment element of the computer program is configured to adjust a functional residual capacity (FRC), a cardiac output (Q), a pulmonary capillary blood flow (Q_{pcbf}), a content of carbon dioxide in venous blood (CᵥCO₂), or any combination thereof input into the algorithmic lung model.

20. The storage medium of claim 16, wherein the collection element of the computer program collects measured data corresponding to an amount of carbon dioxide, oxygen, nitrogen, SF₆, helium, an anesthetic agent in respiration, or any combination thereof of the subject.

21. The storage medium of claim 16, wherein the adjustment element employs a gradient descent technique, an iterative technique, a random search technique, an informed random search technique, or any combination thereof to adjust the value input into the algorithmic lung model.

22. The storage medium of claim 16, wherein the computer program further comprises a perturbation element configured to directly or indirectly cause a respiration of the subject to be perturbated at least once while the measured data is being obtained.
